SEQUENCE LISTING (4 sequences)

SEQ ID NO: 1 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQNAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 2 - protein, 461 aa, Homo sapiens
MYNTVWSMDRDDADWREVMMPYSTELIFYIEMDPPALPPKPPKPMTSAVPNGMKDSSVSLQDAEWYWGDISREEVNDKLRDMPDGTFLVRDASTKMQGDYTLTLRKGGNNKLIKIYHRDGKYGFSDPLTFNSVVELINHYHHESLAQYNPKLDVKLMYPVSRYQQDQLVKEDNIDAVGKKLQEYHSQYQEKSKEYDRLYEEYTRTSQEIQMKRTAIEAFNETIKIFEEQCHTQEQHSKEYIERFRREGNEKEIERIMMNYDKLKSRLGEIHDSKMRLEQDLKKQALDNREIDKKMNSIKPDLIQLRKIRDQHLVWLNHKGVRQKRLNVWLGIKNEDADENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 3 - protein, 728 aa, Homo sapiens
MAGPEGFQYRALYPFRRERPEDLELLPGDVLVVSRAALQALGVAEGGERCPQSVGWMPGLNERTRQRGDFPGTYVEFLGPVALARPGPRPRGPRPLPARPRDGAPEPGLTLPDLPEQFSPPDVAPPLLVKLVEAIERTGLDSESHYRPELPAPRTDWSLSDVDQWDTAALADGIKSFLLALPAPLVTPEASAEARRALREAAGPVGPALEPPTLPLHRALTLRFLLQHLGRVARRAPALGPAVRALGATFGPLLLRAPPPPSSPPPGGAPDGSEPSPDFPALLVEKLLQEHLEEQEVAPPALPPKPPKAKPAPTVLANGGSPPSLQDAEWYWGDISREEVNEKLRDTPDGTFLVRDASSKIQGEYTLTLRKGGNNKLIKVFHRDGHYGFSEPLTFCSVVDLINHYRHESLAQYNAKLDTRLLYPVSKYQQDQIVKEDSVEAVGAQLKVYHQQYQDKSREYDQLYEEYTRTSQELQMKRTAIEAFNETIKIFEEQGQTQEKCSKEYLERFRREGNEKEMQRILLNSERLKSRIAEIHESRTKLEQQLRAQASDNREIDKRMNSLKPDLMQLRKIRDQYLVWLTQKGARQKKINEWLGIKNETEDQYALMEDEDDLPHHEERTWYVGKINRTQAEEMLSGKRDGTFLIRESSQRGCYACSVVVDGDTKHCVIYRTATGFGFAEPYNLYGSLKELVLHYQHASLVQHNDALTVTLAHPVRAPGPGPPPAAR

SEQ ID NO: 4 - protein, 15 aa, Homo sapiens
  MOD_RES (9)..(9): PHOSPHORYLATION; tyrosine at position 9 is phosphorylated
CSKEYDRLYEEYTRT

(19) United States
(12) Patent Application Publication
(10) Pub. No.: US 2008/0014598 A1
(43) Pub. Date: Jan. 17, 2008

(54) PHOSPHO-SPECIFIC ANTIBODIES TO PI3K REGULATORY SUBUNIT AND USES THEREOF

(75) Inventors: Thorsten Wiederhold, Beverly, MA (US); Valerie Goss, Seabrook, NH (US); Albrecht Moritz, Salem, MA (US); Klarisa Rikova, Reading, MA (US); Ting-Lei Gu, Woburn, MA (US); Peter Hornbeck, Magnolia, MA (US)

Correspondence Address: Simona Levi-Minzi, Ph.D.;General Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 11/744,753

(22) Filed: May 4, 2007

Related U.S. Application Data
(60) Provisional application No. 60/833,752, filed on Jul. 27, 2006.
(60) Provisional application No. 60/833,752, filed on Jul. 27, 2006.
(60) Provisional application No. 60/830,550, filed on Jul. 13, 2006.

(51) Int. Cl. G01N 33/573; C07K 16/18; C12N 5/06

(52) U.S. Cl. 435/7.4; 435/326; 530/387.1; 530/388.26; 530/389.1

(57) ABSTRACT

The invention discloses ten newly discovered PI3K regulatory subunit phosphorylation sites, tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma), and provides reagents, including polyclonal and monoclonal antibodies, that selectively bind to PI3K when phosphorylated at one of the disclosed sites. Also provided are assays utilizing this reagent, including methods for determining the phosphorylation of PI3K in a biological sample, selecting a patient suitable for PI3K inhibitor therapy, profiling PI3K activation in a test tissue, and identifying a compound that modulates phosphorylation of PI3K in a test tissue, by using a detectable reagent, such as the disclosed antibody, that binds to PI3K only when phosphorylated at a disclosed site. The sample or test tissue may be taken from a subject suspected of having cancer, such as lymphoma, glioma, and colon cancer, involving altered PI3K signaling.

### RELATED APPLICATIONS

This application claims priority to and the benefit of U.S. Ser. No. 11/503,335, filed Aug. 11, 2006, presently pending, which itself claims priority to PCT/US04/26199, filed Aug. 12, 2004, now abandoned, and U.S. Ser. No. 60/833,752, filed Jul. 27, 2006, presently pending, and PCT/US06/00979, filed Jan. 12, 2006, presently pending, which itself claims priority to U.S. Ser. No. 60/651,583, filed Feb. 10, 2005, now abandoned, and PCT/US04/42940, filed Dec. 21, 2004, presently pending, and PCT/US06/10868, filed Mar. 24, 2006, presently pending, which itself claims priority to U.S. Ser. No. 60/670,447, filed Apr. 12, 2005, now abandoned, and U.S. Ser. No. 60/833,752, filed Jul. 27, 2006, presently pending, and U.S. Ser. No. 60/830,550, filed Jul. 13, 2006, presently pending, the disclosures of which are hereby incorporated herein in their entirety by reference.

### FIELD OF THE INVENTION

The invention relates generally to antibodies, and more particularly to activation state-specific antibodies to receptor tyrosine kinases and their uses.

### BACKGROUND OF THE INVENTION

Many diseases are characterized by disruptions in cellular signaling pathways that lead to pathologies including uncontrolled growth and proliferation of cancerous cells, as well as aberrant inflammation processes. Such defects include changes in the activity of lipid kinases, a class of enzymes that catalyze the transfer of phosphate groups to lipids. These phosphorylated lipids, in turn, recruit important downstream proteins that propagate the signals originating from upstream signaling mediators, such as receptor tyrosine kinase and antigen receptors. For example, the protein kinase Akt is recruited by phospholipids to the plasma membrane where it is activated. Once activated, Akt plays a pivotal role in survival both of normal and cancerous tissues.

Phosphoinositide 3-kinases (PI3Ks) are a family of lipid kinases that play pivotal roles in signaling pathways downstream from multiple cell surface receptors, controlling growth, proliferation, and cell survival. Active PI3Ks consist of two subunits: a regulatory subunit with a molecular weight of either 85 or 55 kD (p85 or p55), and a catalytic subunit of molecular weight 110 kD (p110). While it is clear that the regulatory subunits are critical to the function of PI3K, they also transmit signals independently of PI 3-kinase (Ueki et al., J Biol. Chem. November 28; 278(48): 48453-66 (2003)). It has recently been demonstrated that p85-alpha can induce apoptosis via the inducible transcription factor NFAT3 independent of the PI3K signaling pathway (Song et al., Mol. Cell. Biol. 27: 2713-2731 (2007)).

Three closely related regulatory subunits have been described: p85-alpha (PI3KR1), p85-beta (PI3KR2), and p55-gamma (PI3KR3) (also referred to as PIK3R1-R3). A limited number of phosphorylation sites have previously been reported on PIK3R1 and PIK3R3. The published sites on PIK3R1 are serine 83 (Cosentino et al., Oncogene October O₂ (2006)), tyrosines 368, 580 and 607 (Hayashi et al., J Biol Chem April; 268(10): 7107-17 (1993)), tyrosine 508 (Kavanaugh et al., Biochemistry September 13; 33(36): 11046-50 (1994)), tyrosines 528 and 556 (Kwon et al., Endocrinology March; 147(3): 1458-65 (2006)), serine 608 (Dhand et al., EMBO J. February; 13(3): 522-33 (1994)), and tyrosine 688 (von Willebrand et al., J. Biol. Chem. February; 273(7): 3994-4000 (1998)). The only previously published phosphorylation site on PIK3R3 is tyrosine 341 (Pons et al., Mol Cell Biol. August; 15(8): 4453-65 (1995)). To date, no PI3KR2 phosphorylation sites have been described.

The PI3K pathway is implicated in various human diseases including diabetes, heart failure, and many cancers (see e.g., Kim et al., Curr Opin. Investig. Drugs. December; 6(12): 1250-8 (2005)) including colorectal cancer, acute myeloid leukemia, breast cancer, gliomas, and ovarian cancer. Inhibitors of PI3K are being studied as potential therapeutics in a variety of diseases including cancer, heart failure and autoimmune/inflammatory disorders. For example, the PI3K inhibitor SF1126 is being investigated clinically for the treatment of cancer including multiple myelomas by Semafore Pharmaceuticals, Inc.

Although a limited number of PI3K phosphorylation sites are known, and a few antibodies for their study available, there remains a need for the identification of additional phosphorylation sites relevant to activity of this kinase. Accordingly, new and improved reagents for the detection of PI3K activity would be desirable, including development of reagents against newly identified sites of PI3K phosphorylation. Since phosphorylation-dependent over-activation of PI3K is associated with diseases such as lymphoma, glioma, and colon cancer, reagents enabling the specific detection of PI3K activation would be useful tools for research and clinical applications.

### SUMMARY OF THE INVENTION

The invention discloses ten novel Phosphatidylinositol 3 Kinase (PI3K) regulatory subunit phosphorylation sites, and provides antibodies, both polyclonal and monoclonal, which selectively bind to PI3KR1-R3 only when phosphorylated at one of these novel sites. The novel sites are tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma), occurring on the three paralogs of human PI3K regulatory subunit. Several of the sites (e.g. Tyr 467 in PI3KR1, Tyr 464 in PI3KR2, and Tyr 199 in PI3KR3) are highly homologous across the three paralogs). Also provided are methods of determining the phosphorylation of PI3K in a biological sample, identifying a patient suitable for PI3K inhibitor therapy, profiling PI3K activation in a test tissue, and identifying a compound that modulates phosphorylation of PI3K in a test tissue, by using a detectable reagent, such as the disclosed antibodies, that binds to PI3K when phosphorylated at one of the disclosed sites. In preferred embodiments, the sample or test tissue is taken from a subject suspected of having cancer, such as lymphoma, glioma, and colon cancer, characterized by or involving PI3K activity.

### BRIEF DESCRIPTION OF THE DRAWINGS

FIG. 1—is a multiple sequence alignment showing the amino acid sequences (1-letter code) of human PI3K regulatory subunit paralogs PI3KR1, PI3KR3, and PI3KR2 (SEQ ID NOs: 1-3). Tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma) are shown. Conserved tyrosines presently disclosed are shown in bold. Asterisks indicate amino acid identity between paralogs. The amino acid sequences of these paralogs of PI3K are publicly available at NCBI REFPEPT database (Accession Nos. NP_{—}852664.1, NP_{—}005018.1, NP_{—}003620.2, respectively).

FIG. 2—Western blot analysis of extracts from NIH/3T3-Src cells, untreated or treated with lambda phosphatase and from C2C12 cells, untreated or treated with H2O2, using a phospho-PI3K p85 (Tyr464)/p55 (Tyr199) Antibody (top panel). The same blot was probed with Akt Antibody showing equal loading (bottom panel).

### DETAILED DESCRIPTION OF THE INVENTION

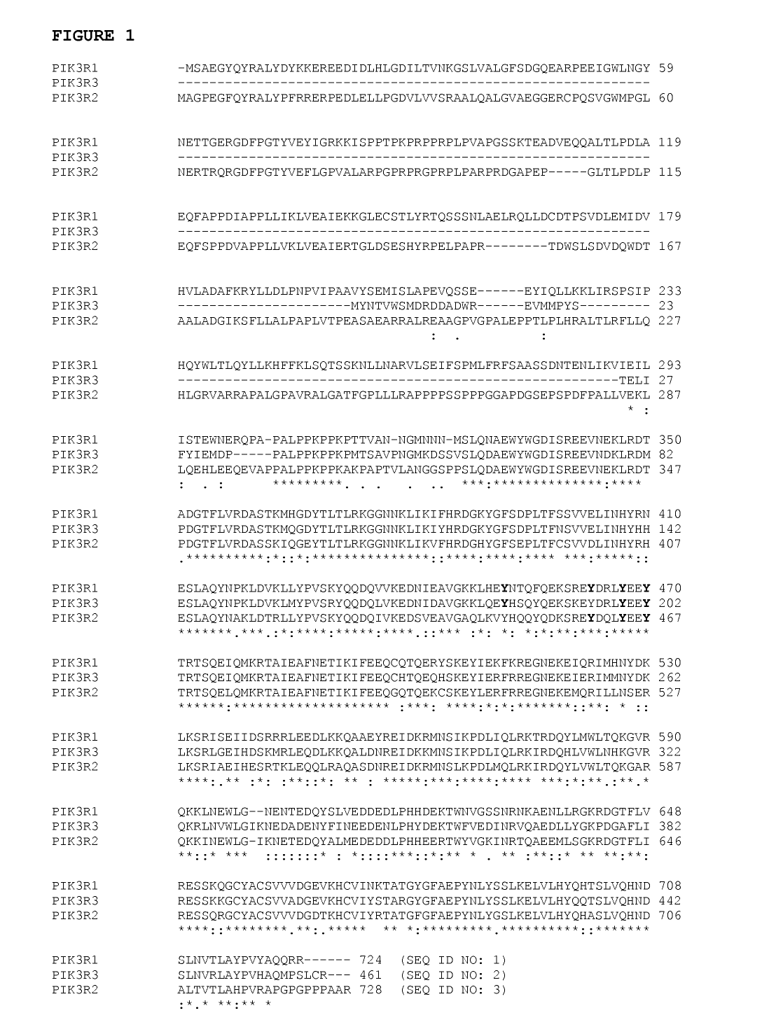

In accordance with the present invention, ten novel phosphorylation sites in human PI3K regulatory subunit have now been identified. The novel sites are tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma), are most are highly homologous phosphorylation sites occurring across these three human PI3K regulatory subunit paralogs (see FIG. 1). The sites each occur in the coiled coil domain of their respective paralog. Although a handful of PI3K regulatory subunit phosphorylation sites have previously been described (see Cosentino et al., supra.; Hayashi et al., supra.; Kavanaugh et al., supra; Kwon et al., supra.; Dhand et al., supra.; von Willebrand et al., supra.; Pons et al., supra.), the ten tyrosine phosphorylation sites disclosed herein are novel.

The newly identified PI3K regulatory subunit phosphorylation sites were first described by the present inventors in U.S. Ser. No. 11/503,335 (Moritz et al.), PCT/US06/00979 (Goss et al.), U.S. Ser. No. 60/651,583 (Guo et al.), PCT/US04/42940 (Guo et al.), PCT/US06/10868 (Guo et al.), U.S. Ser. No. 60/833,752 (Guo et al.), U.S. Ser. No. 60/830,550 (Hornbeck et al.), and were discovered by globally phospho-profiling cellular models of human cancers, including leukemia and carcinoma, using the PhosphoScan® technique described in U.S. Pat. No. 7,198,896, Rush et al., as further described in Example 1 herein. The phospho-profiling identified a total of over 1700 novel tyrosine phosphorylation sites in a multitude of different signaling proteins, including the phosphorylation sites at tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma) presently described.

As a result of this discovery, peptide antigens may now be designed to raise phospho-specific antibodies that bind a PI3K regulatory subunit (paralogs R1-R3) only when phosphorylated at one (or more) of the disclosed phosphorylation sites. These new reagents enable previously unavailable assays for the detection of PI3K phosphorylation at these sites.

The invention provides, in part, phospho-specific antibodies that bind to PI3K regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2), respectively. Also provided are methods of using a detectable reagent that binds to a disclosed phosphorylated PI3K protein to detect PI3K phosphorylation and activation in a biological sample or test tissue suspected of containing phosphorylated PI3K or having altered PI3K activity, as further described below. In a preferred embodiment, the detectable reagent is a PI3K antibody of the invention. All references cited herein are hereby incorporated herein by reference.

### A. Antibodies and Cell Lines

PI3K phosphospecific antibodies of the present invention bind to PI3K regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2), respectively, but do not substantially bind to PI3K when not phosphorylated at these respective sites, nor to PI3K when phosphorylated at other tyrosine residues. The PI3K antibodies of the invention include (a) monoclonal antibody which binds phospho-PI3K sites described above, (b) polyclonal antibodies which bind to phospho-PI3K sites described above, (c) antibodies (monoclonal or polyclonal) which specifically bind to the phospho-antigen (or more preferably the epitope) bound by the exemplary PI3K phospho-specific antibodies disclosed in the Examples herein, and (d) fragments of (a), (b), or (c) above which bind to the antigen (or more preferably the epitope) bound by the exemplary antibodies disclosed herein. Such antibodies and antibody fragments may be produced by a variety of techniques well known in the art, as discussed below. Antibodies that bind to the phosphorylated epitope (i.e., the specific binding site) bound by the exemplary PI3K antibodies of the Examples herein can be identified in accordance with known techniques, such as their ability to compete with labeled PI3K antibodies in a competitive binding assay.

The preferred epitopic site of the PI3K antibodies of the invention is a peptide fragment consisting essentially of about 11 to 17 amino acids comprising a phosphorylated tyrosine site described herein (tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2), respectively), wherein about 5 to 8 amino acids are positioned on each side of the tyrosine phosphorylation site (for example, residues 194-203 of SEQ ID NO: 2).

The invention is not limited to PI3K antibodies, but includes equivalent molecules, such as protein binding domains or nucleic acid aptamers, which bind, in a phospho-specific manner, to essentially the same phosphorylated epitope to which the PI3K antibodies of the invention bind. See, e.g., Neuberger et al., Nature 312: 604 (1984). Such equivalent non-antibody reagents may be suitably employed in the methods of the invention further described below.

The term “antibody” or “antibodies” as used herein refers to all types of immunoglobulins, including IgG, IgM, IgA, IgD, and IgE, including Fab or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The term “PI3K antibodies” means phospho-specific antibodies that selectively PI3K regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2), respectively, both monoclonal and polyclonal, as disclosed herein. The term “does not bind” with respect to such antibodies means does not substantially react with as compared to binding to phospho-PI3K. The antibodies may bind the regulatory subunit alone or when complexed with the catalytic subunit to form the complete PI3K holoenyzme.

The term “detectable reagent” means a molecule, including an antibody, peptide fragment, binding protein domain, etc., the binding of which to a desired target is detectable or traceable. Suitable means of detection are described below.

Polyclonal antibodies of the invention may be produced according to standard techniques by immunizing a suitable animal (e.g., rabbit, goat, etc.) with an antigen encompassing a PI3K phosphorylation site described herein, collecting immune serum from the animal, and separating the polyclonal antibodies from the immune serum, in accordance with known procedures. In a preferred embodiment, the antigen is a phospho-peptide antigen comprising the site sequence surrounding and including the respective phosphorylated tyrosine residue described herein, the antigen being selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). An exemplary peptide antigen, CSKEYDRLyEEYTRT (where y=phosphotyrosine) (SEQ ID NO: 4) for PI3K p55 (Tyr199) is described in the Examples, below. It will be appreciated by those of skill in the art that longer or shorter phosphopeptide antigens may be employed. See Id. Polyclonal PI3K antibodies produced as described herein may be screened as further described below.

Monoclonal antibodies of the invention may be produced in a hybridoma cell line according to the well-known technique of Kohler and Milstein. Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The invention also provides hybridoma clones, constructed as described above, that produce PI3K monoclonal antibodies of the invention. Similarly, the invention includes recombinant cells producing a PI3K antibody as disclosed herein, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

PI3K antibodies of the invention, whether polyclonal or monoclonal, may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e. that epitope including a tyrosine phosphorylation site disclosed herein) and for reactivity only with the phosphorylated form of the antigen. Peptide competition assays may be carried out to confirm lack of reactivity with other PI3K phospho-epitopes. The antibodies may also be tested by Western blotting against cell preparations containing PI3K, e.g. cell lines over-expressing PI3K, to confirm reactivity with the desired phosphorylated target.

Specificity against the desired phosphorylated epitopes may also be examined by construction PI3K mutants lacking phosphorylatable residues at positions outside the desired epitope known to be phosphorylated, or by mutating the desired phospho-epitope and confirming lack of reactivity. PI3K antibodies of the invention may exhibit some cross-reactivity with non-PI3K epitopes. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-PI3K proteins is readily characterized by Western blotting alongside markers of known molecular weight. Amino acid sequences of cross-reacting proteins may be examined to identify sites highly homologous to a PI3K sequence surrounding any of the phosphorylated tyrosines disclosed herein.

In certain cases, polyclonal antisera may be exhibit some undesirable general cross-reactivity to phosphotyrosine, which may be removed by further purification of antisera, e.g. over a phosphotyramine column. PI3K phospho-specific antibodies raised against one of the disclosed subunit paralog phosphorylation sites may also cross-react with one or more of the nearly identical sites in the other paralogs, as expected. For example, a phospho-specific antibody raised against the PI3KR2 (Tyr464) site may cross-react with the nearly-identical PI3KR3 (Tyr199) site, which differ by only two amino acids.

PI3K antibodies may be further characterized via immunohistochemical (IHC) staining using normal and diseased tissues to examine PI3K phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

### B. Detection & Profiling Methods

The methods disclosed herein may be employed with any biological sample suspected of containing phosphorylated PI3K, and in particular, PI3K regulatory subunit phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2). Biological samples taken from human subjects for use in the methods disclosed herein are generally biological fluids such as serum, blood plasma, fine needle aspirate, ductal lavage, bone marrow sample or ascites fluid. In the alternative, the sample taken from the subject can be a tissue sample (e.g., a biopsy tissue), such as tumor tissue.

In one embodiment, the invention provides a method for detecting phosphorylated PI3K in a biological sample by (a) contacting (binding) a biological sample suspected of containing phosphorylated PI3K with at least one antibody that binds to a Phosphatidylinositol 3 Kinase (PI3K) regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2) under conditions suitable for formation of a reagent-PI3K complex, and (b) detecting the presence of the complex in the sample, wherein the presence of the complex indicates the presence of phosphorylated PI3K in the sample. Biological samples may be obtained from subjects suspected of having a disease involving altered PI3K expression or activity (e.g., lymphoma, glioma, colon cancer, lung cancer, and ovarian cancer). Samples may be analyzed to monitor subjects who have been previously diagnosed as having cancer, to screen subjects who have not been previously diagnosed as carrying cancer, or to monitor the desirability or efficacy of therapeutics targeted at PI3K. Subjects may be either children or adults. In the case of colon cancer, for example, the subjects will most frequently be adult males.

In another embodiment, the invention provides a method for profiling PI3K activation in a test tissue suspected of involving altered PI3K activity, by (a) contacting the test tissue with at least one antibody that binds to a PI3K regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2) under conditions suitable for formation of a reagent-PI3K complex, (b) detecting the presence of the complex in the test tissue, wherein the presence of the complex indicates the presence of phosphorylated PI3K in the test tissue, and (c) comparing the presence of phosphorylated PI3K detected in step (b) with the presence of phosphorylated PI3K in a control tissue, wherein a difference in PI3K phosphorylation profiles between the test and control tissues indicates altered PI3K activation in the test tissue. In a preferred embodiment, the reagent is a PI3K antibody of the invention. In other preferred embodiments, the test tissue is a cancer tissue, such as lymphoma, glioma, and colon cancer tissue, suspected of involving altered PI3K phosphorylation.

The methods described above are applicable to examining tissues or samples from PI3K related cancers, particularly colorectal cancer, acute myeloid leukemia, breast cancer, gliomas, and ovarian cancer, in which phosphorylation of PI3K at any of the novel sites disclosed herein has predictive value as to the outcome of the disease or the response of the disease to therapy. It is anticipated that the PI3K antibodies will have diagnostic utility in a disease characterized by, or involving, altered PI3K activity or altered PI3K phosphorylation. The methods are applicable, for example, where samples are taken from a subject has not been previously diagnosed as having lymphoma, glioma, and colon cancer, nor has yet undergone treatment for lymphoma, glioma, and colon cancer, and the method is employed to help diagnose the disease, monitor the possible progression of the cancer, or assess risk of the subject developing such cancer involving PI3K phosphorylation. Such diagnostic assay may be carried out prior to preliminary blood evaluation or surgical surveillance procedures.

Such a diagnostic assay may be employed to identify patients with activated PI3K who would be most likely to respond to cancer therapeutics targeted at inhibiting PI3K activity. Such a selection of patients would be useful in the clinical evaluation of efficacy of existing or future PI3K inhibitors, as well as in the future prescription of such drugs to patients. Accordingly, in another embodiment, the invention provides a method for selecting a patient suitable for PI3K inhibitor therapy, said method comprising the steps of (a) obtaining at least one biological sample from a patient that is a candidate for PI3K inhibitor therapy, (b) contacting the biological sample with at least one PI3K phospho-specific antibody described herein under conditions suitable for formation of a reagent-PI3K complex, and (c) detecting the presence of the complex in the biological sample, wherein the presence of said complex indicates the presence of phosphorylated PI3K in said test tissue, thereby identifying the patient as potentially suitable for PI3K inhibitor therapy.

Alternatively, the methods are applicable where a subject has been previously diagnosed as having, e.g. lymphoma, glioma, and colon cancer, and possibly has already undergone treatment for the disease, and the method is employed to monitor the progression of such cancer involving PI3K phosphorylation, or the treatment thereof.

In another embodiment, the invention provides a method for identifying a compound which modulates phosphorylation of PI3K in a test tissue, by (a) contacting the test tissue with the compound, (b) detecting the level of phosphorylated PI3K in said the test tissue of step (a) using at least one PI3K phospho-specific antibody described herein under conditions suitable for formation of an antibody-PI3K complex, and (c) comparing the level of phosphorylated PI3K detected in step (b) with the presence of phosphorylated PI3K in a control tissue not contacted with the compound, wherein a difference in PI3K phosphorylation levels between the test and control tissues identifies the compound as a modulator of PI3K phosphorylation. In some preferred embodiments, the test tissue is a taken from a subject suspected of having cancer and the compound is a PI3K inhibitor. The compound may modulate PI3K activity either positively or negatively, for example by increasing or decreasing phosphorylation or expression of PI3K. PI3K phosphorylation and activity may be monitored, for example, to determine the efficacy of an anti-PI3K therapeutic, e.g. a PI3K inhibitor.

Conditions suitable for the formation of antibody-antigen complexes or reagent-PI3K complexes are well known in the art (see part (d) below and references cited therein). It will be understood that more than one PI3K antibody may be used in the practice of the above-described methods. For example, PI3KR1 (PI3Kp85 alpha) (Tyr467) phospho-specific antibody and a PI3KR2 (PI3Kp85 beta) (Tyr460) phospho-specific antibody may be simultaneously employed to detect phosphorylation of both tyrosines in these two subunit paralogs in one step. Alternatively, multiple antibodies may be simultaneously employed to detect phosphorylation of multiple tyrosines on a single subunit paralog in one step.

### C. Immunoassay Formats & Diagnostic Kits

Assays carried out in accordance with methods of the present invention may be homogeneous assays or heterogeneous assays. In a homogeneous assay the immunological reaction usually involves a PI3K-specific reagent (e.g. a PI3K antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a PI3K-specific reagent (e.g., the PI3K antibody of the invention), and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of phosphorylated PI3K is detectable compared to background.

PI3K antibodies disclosed herein may be conjugated to a solid support suitable for a diagnostic assay (e.g., beads, plates, slides or wells formed from materials such as latex or polystyrene) in accordance with known techniques, such as precipitation. Antibodies of the invention, or other PI3K binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

PI3K antibodies of the invention may also be optimized for use in a flow cytometry assay to determine the activation status of PI3K in patients before, during, and after treatment with a drug targeted at inhibiting PI3K phosphorylation at a tyrosine site disclosed herein. For example, bone marrow cells or peripheral blood cells from patients may be analyzed by flow cytometry for PI3K phosphorylation, as well as for markers identifying various hematopoietic cell types. In this manner, PI3K activation status of the malignant cells may be specifically characterized.

Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary PI3K antibody, washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated PI3K in the malignant cells and reveal the drug response on the targeted PI3K protein.

Alternatively, PI3K antibodies of the invention may be optimized for use in other clinically-suitable applications, for example bead-based multiplex-type assays, such as IGEN, Luminex™ and/or Bioplex™ assay formats, or otherwise optimized for antibody arrays formats.

Diagnostic kits for carrying out the methods disclosed above are also provided by the invention. Such kits comprise at least one detectable reagent that binds to PI3K when phosphorylated at a novel tyrosine phosphorylation site disclosed herein (a phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma)). In a preferred embodiment, the reagent is a PI3K antibody of the invention. In one embodiment, the diagnostic kit comprises (a) a PI3K antibody of the invention conjugated to a solid support and (b) a second antibody conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. In another embodiment a kit (e.g. a kit for the selection of a patient suitable for PI3K inhibitor therapy) comprises (a) a PI3K antibody as described herein, and (b) a specific binding partner (i.e. secondary antibody) conjugated to a detectable group.

The primary (phospho-PI3K) detection antibody may itself be directly labeled with a detectable group, or alternatively, a secondary antibody, itself labeled with a detectable group, that binds to the primary antibody may be employed. Labels (including dyes and the like) suitable as detectable agents are well known in the art. Ancillary agents as described above may likewise be included. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Identification of Novel PI3K Regulatory Subunit Phosphorylation Sites by Global Phospho-Profiling

In order to discover previously unknown signal transduction protein phosphorylation sites, PhosphoScan® peptide isolation and characterization techniques (as described in U.S. Pat. No. 7,198,896, Rush et al.) were employed to identify phosphotyrosine-containing peptides in cell extracts from several dozen human cancer lines, including leukemia and carcinoma cell lines. This work was first described by the present inventors in U.S. Ser. No. 11/503,335 (Moritz et al.), PCT/US06/00979 (Goss et al.), U.S. Ser. No. 60/651,583 (Guo et al.), PCT/US04/42940 (Guo et al.), PCT/US06/10868 (Guo et al.), U.S. Ser. No. 60/833,752 (Guo et al.), U.S. Ser. No. 60/830,550 (Hornbeck et al.), the disclosures of which are incorporated herein by reference in their entirety.

Briefly, tryptic phosphotyrosine-containing peptides were purified and analyzed from extracts of each of cancer cell lines as follows. Cells were cultured in DMEM medium or RPMI 1640 medium supplemented with 10% fetal bovine serum and penicillin/streptomycin. Cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM 9-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) was coupled at 4 mg/ml beads to protein G or protein A agarose (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitrile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LCQ Deca XP Plus ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (either as released on Apr. 29, 2003 and containing 37,490 protein sequences or as released on Feb. 23, 2004 and containing 27,175 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. Assigned sequences were accepted or rejected following a conservative, two-step process. In the first step, a subset of high-scoring sequence assignments was selected by filtering for XCorr values of at least 1.5 for a charge state of +1, 2.2 for +2, and 3.3 for +3, allowing a maximum RSp value of 10. Assignments in this subset were rejected if any of the following criteria were satisfied: (i) the spectrum contained at least one major peak (at least 10% as intense as the most intense ion in the spectrum) that could not be mapped to the assigned sequence as an a, b, or y ion, as an ion arising from neutral-loss of water or ammonia from a b or y ion, or as a multiply protonated ion; (ii) the spectrum did not contain a series of b or y ions equivalent to at least six uninterrupted residues; or (iii) the sequence was not observed at least five times in all the studies we have conducted (except for overlapping sequences due to incomplete proteolysis or use of proteases other than trypsin). In the second step, assignments with below-threshold scores were accepted if the low-scoring spectrum showed a high degree of similarity to a high-scoring spectrum collected in another study, which simulates a true reference library-searching strategy. All spectra supporting the final list of 424 assigned sequences identified (data not shown) were reviewed by at least three people to establish their credibility.

The phospho-profiling of the examined cell lines identified a total of over 1700 novel tyrosine phosphorylation sites in a multitude of different signaling proteins, including the phosphorylation sites at tyrosines 467, 452, 463, and 470 in PI3KR1 (PI3Kp85 alpha), tyrosines 464, 460, and 467 in PI3KR2 (PI3Kp85 beta), and tyrosines 199, 184, and 202 in PI3KR3 (PI3Kp55 gamma) presently described.

### EXAMPLE 2

### Development of the Phospho-PI3K p85 (Tyr458)/p55 (Tyr199) Polyclonal Antibody

A 15 amino acid phospho-peptide antigen, CSKEYDRLyEEYTRT (where y=phosphotyrosine) (SEQ ID NO: 4), corresponding to residues 192-205 of human PI3K p55 encompassing the tyrosine 199 plus cysteine on the N-terminus for coupling, was constructed according to standard synthesis techniques using a Rainin/Protein Technologies, Inc., Symphony peptide synthesizer. See ANTIBODIES: A LABORATORY MANUAL, supra.; Merrifield, supra.

These peptides were coupled to KLH, and rabbits are then injected intradermally (ID) on the back with antigen in complete Freunds adjuvant (500 μg antigen per rabbit). The rabbits were boosted with the same antigen in incomplete Freund adjuvant (250 μg antigen per rabbit) every three weeks. After the fifth boost, the bleeds were collected. The sera were purified by Protein A-affinity chromatography as previously described (see ANTIBODIES: A LABORATORY MANUAL, Cold Spring Harbor, supra.). The eluted immunoglobulins are then loaded onto a resin-CSKEYDRLyEEYTRT Knotes column. After washing the column extensively, the phospho-PI3K p85 (Tyr458)/p55 (Tyr199) antibodies were eluted and kept in antibody storage buffer.

The antibody was further tested for phospho-specificity by Western blot analysis. NIH/3T3 and C2C12 cells may be obtained from ATCC in Manassas, Va. NIH/3T3 cells were transfected with src and stable clones were selected using puromycin. NIH/3T3-src cells are cultured in DMEM supplemented with 10% CS and 1.5 μg/ml puromycin. NIH/3T3-src cells were treated with X protein phosphatase (0 units/ml vs. 4000 units/ml) for 1 h at 37 C, washed with PBS and lysed. C2C12 cells are cultured DMEM supplemented with 10% FBS. C2C12 cells were stimulated with H₂O₂ (0 μM vs. 50 μM) for 20 minutes at 37 C, washed with PBS and directly lysed in cell lysis buffer. Loading buffer was added to all cell lysates and the mixture was boiled for 5 minutes. 20 μl (˜20 μg protein) of sample was loaded onto an 8% SDS-PAGE gel.

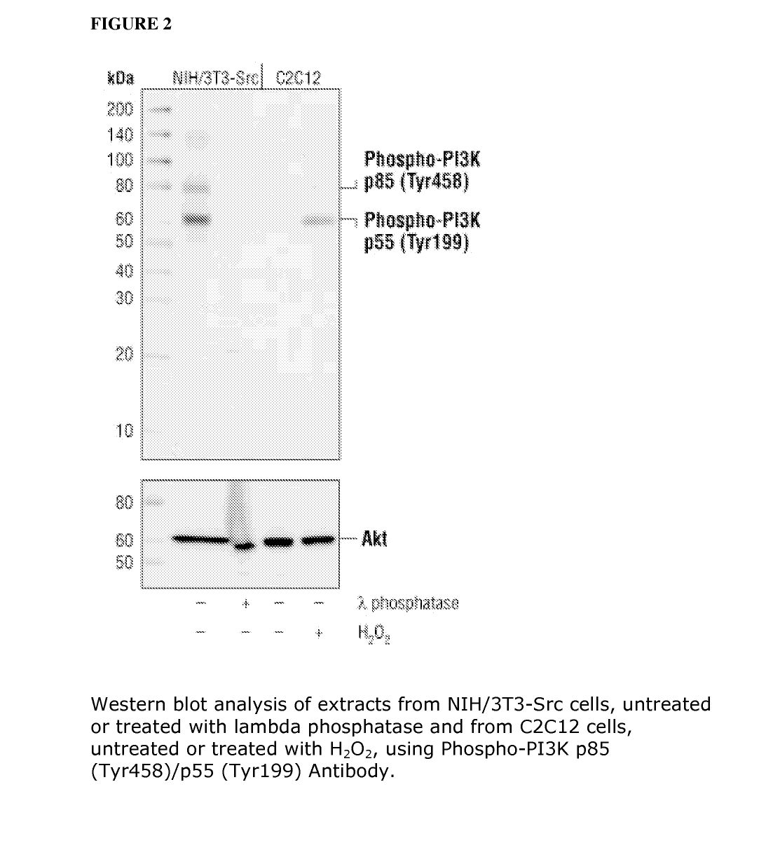

A standard Western blot was performed according to the Immunoblotting Protocol set out in the Cell Signaling Technology 2005-06 Catalogue and Technical Reference, p. 415. The phospho-PI3K p85 (Tyr458)/p55 (Tyr199) polyclonal antibody is used at dilution 1:1000 (for further details see product #4228 at www.cellsignal.com). The results of the Western blot—see FIG. 2—show that the antibody, only recognizes a ˜85 kDa phospho-protein (phospho-PI3K p85 (Tyr458)) and a ˜55 kDa phospho-protein (phospho-PI3K p55 (Tyr199)) activated by Src or H₂O₂. The antibody does not recognize the non-tyrosine phosphorylated PI3K p85 (Tyr458)/p55 (Tyr199) in X protein phosphatase treated NIH/3T3-src or non-stimulated C2C12 cells.

### EXAMPLE 3

### Production of a Phospho-PI3K p55 (Tyr199) Phosphospecific Monoclonal Antibody

A PI3K p55 (Tyr199) phosphospecific rabbit monoclonal antibody, may be produced from spleen cells of the immunized rabbit described in Example 2, above, following standard procedures (Harlow and Lane, 1988). The rabbit splenocytes are fused to proprietary fusion partner cells according to a standard protocol (see generally Loyola School of Medicine protocol (Helga Spieker-Polet) at http://www.meddean.luc.edu/lumen/DeptWebs/microbio/KNIGHT/PROTOC/Hybridom.htm.)

Colonies originating from the fusion may be screened by ELISA for reactivity to the phospho-peptide and non-phospho-peptide and by Western blot analysis. Colonies found to be positive by ELISA to the phospho-peptide while negative to the non-phospho-peptide are further characterized by Western blot analysis. Colonies found to be positive by Western blot analysis are then subcloned by limited dilution. Rabbit ascites are produced from the single clone obtained from subcloning.

Specificity may be determined by Western Blot as described in Example 2 above, using non-tyrosine phosphorylated PI3K p85 (Tyr458)/p55 (Tyr199) in λ protein phosphatase treated NIH/3T3-src or non-stimulated C2C12 cells for a negative control. Rabbit monoclonal antibody raised to PI3K p55 (Tyr199) is expected to cross-react with the nearly-identical PI3K p85 (Tyr458) site, as described above in Example 2 for the polyclonal antibody.

### EXAMPLE 4

### Detection of PI3K Phosphorylation in Cytometric Assay

The PI3K phosphospecific antibodies described in Examples 2 or 3 may be used in flow cytometry to detect phospho-PI3K in a biological sample. A sample of cells may be taken to be analyzed by Western blot analysis. The remaining cells are fixed with 1% paraformaldehyde for 10 minutes at 37° C., followed by cell permeabilization 90% with methanol for 30 minutes on ice. The fixed cells are then stained with the phospho-PI3K primary antibody for 60 minutes at room temperature. The cells are then washed and stained with an Alexa 488-labeled secondary antibody for 30 minutes at room temperature. The cells may then be analyzed on a Beckman Coulter EPICS-XL flow cytometer.

The cytometric results are expected to match the Western results described above, further demonstrating the specificity of the PI3K antibody for the activated/phosphorylated PI3K protein.

### EXAMPLE 5

### Detection of Constitutively Active PI3K in Cells Using Flow Cytometry

PI3K phosphospecific antibody described in Examples 2 or 3 above may also be used in flow cytometry to detect phospho-PI3K in a biological sample. Serum-starved cells may be incubated with or without a PI3K inhibitor SF1126 for 4 hours at 37° C. The cells are then fixed with 2% paraformaldehyde for 10 minutes at 37° C. followed by cell permeabilization 90% with methanol for 30 minutes on ice. The fixed cells are stained with the Alexa 488-conjugated PI3K primary antibody for 1 hour at room temperature. The cells may then be analyzed on a Beckman Coulter EPICS-XL flow cytometer.

The cytometric results are again expected to demonstrate the specificity of the PI3K antibody for the activated PI3K protein and the assay's ability to detect the activity and efficacy of a PI3K inhibitor. In the presence of the drug, a population of the cells will show less staining with the antibody, indicating that the drug is active against PI3K.

What is claimed is:

1. An isolated antibody that binds to a Phosphatidylinositol 3 Kinase (PI3K) regulatory subunit only when phosphorylated at a tyrosine phosphorylation site selected from the group consisting of tyrosines 467, 452, 463, and 470 in PI3KR1 (PI3Kp85 alpha) (SEQ ID NO: 1), tyrosines 464, 460, and 467 in PI3KR2 (PI3Kp85 beta) (SEQ ID NO: 3), and tyrosines 199, 184, and 202 in PI3KR3 (PI3Kp55 gamma) (SEQ ID NO: 2).

2. The antibody of claim 1, wherein said antibody is polyclonal.

3. The antibody of claim 1, wherein said antibody is monoclonal.

4. A hybridoma cell line producing the antibody of claim 3.

5. The hybridoma cell line of claim 4, wherein said cell line is a rabbit hybridoma or a mouse hybridoma.

6. A monoclonal antibody produced by the hybridoma cell line of claim 5.

7. A method for detecting phosphorylated PI3K in a biological sample, said method comprising the steps of:
(a) contacting a biological sample suspected of containing phosphorylated PI3K with at least one antibody of claim 1 under conditions suitable for formation of an antibody-PI3K complex; and
(b) detecting the presence of said complex in said sample, wherein the presence of said complex indicates the presence of phosphorylated PI3K in said sample.

8. The method of claim 7, wherein said biological sample is taken from a subject suspected of having cancer.

9. A method of identifying a compound that modulates phosphorylation of PI3K in a test tissue, said method comprising the steps of:
(a) contacting said test tissue with said compound;
(b) detecting the level of phosphorylated PI3K in said test tissue of step (a) using at least one antibody of claim 1 under conditions suitable for formation of a antibody-PI3K complex;
(c) comparing the level of phosphorylated PI3K detected in step (b) with the presence of phosphorylated PI3K in a control tissue not contacted with said compound, wherein a difference in PI3K phosphorylation levels between said test tissue and said control tissue identifies said compound as a modulator of PI3K phosphorylation.

10. The method of claim 9, wherein said test tissue is taken from a subject suspected of having cancer.

11. The method of claim 9, wherein said compound is a PI3K inhibitor.

12. A kit for the detection of phosphorylated PI3K in a biological sample, said kit comprising (a) at least one detectable antibody of claim 1, and (b) at least one secondary reagent.
